(19) United States
(12) Patent Application Publication
(10) Pub. No.: US 2023/0040010 A1
(43) Pub. Date: Feb. 9, 2023

(54) COMPOSITIONS AND METHODS FOR THE TREATMENT OF MASTITIS

(71) Applicant: SOCIETE DES PRODUITS NESTLE S.A., Vevey (CH)

(72) Inventors: MYRIAM AFEICHE ZEHIL, Lausanne 26 (CH); TINU MARY SAMUEL, La Tour-de-Peilz (CH); CARLOS ANTONIO DE CASTRO, Singapore (SG)

(21) Appl. No.: 17/758,809

(22) PCT Filed: Jan. 18, 2021
(86) PCT No.: PCT/EP2021/050891

§ 371 Date: Jul. 14, 2022

(30) Foreign Application Priority Data
Jan. 16, 2020 (EP) 20152301.6

(51) Int. Cl. A23L 33/00; A23L 33/155; A23L 33/125; A23L 33/105; A23L 33/16; A23L 33/12; A23L 33/19

(52) U.S. Cl. CPC A23L 33/40; A23L 33/12; A23L 33/16; A23L 33/19; A23L 33/105; A23L 33/125; A23L 33/155; A23V 2002/00

(57) ABSTRACT

The present invention relates to compositions for use in treating or preventing mastitis, for example sub-clinical mastitis, in a subject. In particular, the invention relates to a diet and /or nutritional composition which are characterised by a low inflammatory potential for their use in in treating or preventing mastitis, in particular sub-clinical mastitis, in lactating women. The present invention also relates to a risk prediction tool for the occurrence of subclinical mastitis in lactating women, which is based on the inflammatory potential of their diets.

### FIELD OF THE INVENTION

The present invention relates to compositions for use in treating or preventing mastitis, for example sub-clinical mastitis, in a subject. In particular, the invention relates to a diet and/or to food parameters, or composition comprising such nutrients, which are characterised by a low inflammatory potential for their use in in treating or preventing mastitis, in particular sub-clinical mastitis, in lactating women. The present invention also relates to a risk prediction tool for the occurrence of mastitis, for example subclinical mastitis, in lactating women, which is based on the inflammatory potential of their diets.

### BACKGROUND TO THE INVENTION

WHO recommends that infants should be exclusively breastfed for the first six months of life to achieve optimal growth, development and health and continued breast feeding until 2 years of age. According to WHO, exclusive breastfeeding means that the infant receives only breast milk (no other liquids or solids are given - not even water - with the exception of oral rehydration solution, or drops/syrups of vitamins, minerals or medicines). WHO also recommends early initiation of breastfeeding as this may is critical to newborn survival and to establishing breastfeeding over the long term.

Mastitis is an inflammation of the mammary gland tissue, which can be classified as sub-clinical or clinical depending on the degree of inflammation.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain. Studies among different ethnicities have reported varying estimates of SCM prevalence reaching to as high as 66% with most of the cases occurring in early lactation and typically declining over time.

Staphylococcus infections, in particular S. aureus and S. epidermidis infections, are understood to be a primary cause of mastitis.

Mastitis can result in curtailment or even lack of initiation of breast-feeding of an infant.

Furthermore, the composition of breast milk may change during mastitis, for example increasing in content of sodium and inflammatory mediators, which may adversely affect the nutrition provided to the infant.

Current treatment of mastitis typically involves the administration of antibiotics. However, wide-spread use of antibiotics presents several challenges, including ineffectiveness due to antibiotic resistance, the creation of multiple-antibiotic resistant strains of bacteria, the formation of biofilms, vaginal candidiasis and antibiotic-associated diarrhoea.

Moreover, it has been indicated that there is insufficient evidence to support the effectiveness of antibiotic therapy for the treatment of lactational mastitis (Jahanfar, S. et al. (2013) Cochrane Database Syst Rev 28: CD005458).

Additionally, there are no existing solutions to address the sub-clinical form of mastitis, which is associated with elevated lactation failure and can quickly progresses to Mastitis.

Accordingly, there is a significant need for a way of predicting the risk of occurrence for mastitis, especially subclinical mastitis and/or for alternative or improved methods of treating and preventing mastitis and/or subclinical mastitis.

The present invention address the important issue of addressing the subclinical form of mastitis, while identifying women at risk at an earlier stage before progression in to full-blown mastitis and thereby helping them continue breastfeeding effectively.

### SUMMARY OF THE INVENTION

The inventors have surprisingly found that occurrence and severity of subclinical mastitis correlates with increasing inflammatory potential of the diet (reflected by the overall Dietary Inflammatory Index score) in a population of lactating women.

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

The inventors have studied the dietary intake of women with Na:K ratios greater than 0.6 and compared this to the dietary intake of normal women (without SCM). Differences have been found in terms of median (and mean) of overall Dietary Inflammatory Index score for in the diet followed by such women.

Specifically, the inventors have found that women with sub-clinical mastitis (moderate or severe, as indicated by the respective Na:K ratio) had followed a diet characterized by a higher inflammatory potential as compared to the diet followed by women with no SCM. The inflammatory potential of the diet (overall Dietary Inflammatory Index Score) has been calculated following the methodology described in Shivappa et al, Public Health Nutrition, 17 (8), 1689-1696 (hereafter also referred to as “Shivappa et al. publication”) in the form of a literature derived population based dietary inflammatory index. The whole content of such scientific publication is incorporated by reference into the present patent application.

This indicates that a diet with low inflammatory potential as provided by an overall dietary inflammatory index score lower than 0.8 (such overall dietary inflammatory index score being calculated according to methodology described in Shivappa et al, Public Health Nutrition, 17 (8), 1689-1696) can prevent and/or treat mastitis, in particular subclinical mastitis in lactating women.

In one aspect, the present invention provides for a diet with an overall Dietary Inflammatory index score lower than 0.8 for the prevention and/or treatment of mastitis, for example subclinical mastitis, in a subject, for example a lactating woman.

In another aspect, the present invention provides for a method for prevention and/or treatment of mastitis, for example subclinical mastitis, in a subject, for example a lactating woman, comprising administering to such subject a diet which has an overall Dietary Inflammatory index score lower than 0.8.

In another aspect, the present invention provides for a food parameter having a food parameter-specific overall inflammatory effect score lower than 0 for prevention and/or treatment of mastitis, for example subclinical mastitis.

In another aspect, the present invention provides for a method for prevention and/or treatment of mastitis, for example subclinical mastitis, in a subject, for example a lactating woman, comprising administering to such subject a food parameter which has a food parameter-specific overall inflammatory effect score lower than 0.

In a further aspect, the present invention provides for a food parameter which has a food parameter-specific overall inflammatory effect score lower than 0 for the manufacture of a nutritional composition for the prevention and/or treatment of mastitis, for example subclinical mastitis.

In an additional aspect, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) using the dietary intake data recorded in step a);
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 and/or to consume food parameters, or compositions comprising them, having a food parameter-specific overall inflammatory effect score lower than 0; and/or
e) optionally recommending to the subject to limit the consumption of food parameters, or compositions comprising them, having a food parameter-specific overall inflammatory effect score higher than 0.

In one embodiment, such method may be implemented on a computer.

In another aspect, the present invention provides for a data processing device comprising means for carrying out a computer implemented method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) ) using the dietary intake data recorded in step a);
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

In a still further aspect, the present invention provides for a computer readable medium comprising instructions which, when executed by a computer, cause the computer to carry out the steps of a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculating the overall Dietary Inflammatory Index score with the procedure described in Shivappa et al, Public Health Nutrition, 17 (8), 1689-1696) using the dietary intake data recorded in step a);
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to avoid nutrients and/or foods, or compositions comprising them, having a food parameter-specific overall inflammatory effect score higher than 0.

In a further aspect of the present invention, a nutritional composition is provided comprising at least one food parameter having a food parameter-specific overall inflammatory effect score lower than 0 for the prevention of treatment of mastitis, for example subclinical mastitis.

### BRIEF DESCRIPTION OF THE DRAWINGS

Reference FIG. 1 corresponds to FIG. 1 in the Shivappa et al. publication and is hereby reported for easy of reference of the steps performed in Example 1 for the calculation of the overall Dietary Inflammatory Index score for the Dietsof the study population.

FIG. 2 is graphical representation of the Dietary Inflammatory Index cut off values at the X axis and the relative risk of SCM incidence based on this cutoff point at the Y axis.

### DETAILED DESCRIPTION OF THE INVENTION

### Definitions

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Within the context of the present invention, the term “computer implemented method” indicates a method wherein the steps can be carried out by computer program instructions running on means which provide generic data processing function. Such means can be embedded, for example in a personal computer, a smartphone, a tablet, etc

Within the context of the present invention, the expression “Shivappa et al.” or “Shivappa et al. publication ” refers to the scientific publication Shivappa et al, Public Health Nutrition, 17 (8), 1689-1696.

Within the context of the present invention, the term “overall Dietary Inflammatory Index score” or “Dietary inflammatory Index” or “DII” indicates an index which can be associated to the diet of an individual and which indicates the inflammatory potential of such diet. The Dietary Inflammatory Index is calculated according to the methodology described in Shivappa et al, Public Health Nutrition, 17 (8), 1689-1696.

Within the context of the present invention, the term “food parameter” indicates a whole food, nutrient and/or bioactive comprised in food. Within the meaning of such term, also “ingredients” and “ingredients providing nutrient X” are comprised.

In one embodiment of the present invention, the food parameter is a nutrient or an ingredient comprising and/or providing such nutrient.

Within the context of the present invention, the term “food parameter-specific overall inflammatory effect score” or “food overall inflammatory effect score” indicates an index which can be associated to an individual whole food nutrient and/or bioactive comprised in food and which indicates the inflammatory potential of such whole food, nutrient and/or bioactive comprised in food. The food parameter-specific overall inflammatory effect score is calculated according to the methodology described in Shivappa et al, Public Health Nutrition, 17 (8), 1689-1696 and it is reported in Table 2 of such publication for 45 food parameters.

Within the context of the present invention, the term "nutrient" or nutrients" is intended to comprise either macronutrients (for example carbohydrates, proteins or fats) and/or micronutrients (for example minerals or vitamins) for the human body. Within the context of the present invention, the term “Diet” indicates the total food consumption of a subject on a daily basis. For example, the subject can be a lactating woman.

Within the context of the present invention, the term “ingredient” or “ingredients” indicates an edible substance or mixture of substances which comprises or is essentially consisting of a nutrient for the human body.

In one embodiment of the present invention, the term “ingredient” or “ingredients” indicates an edible substance essentially consisting of a nutrient for the human body.

Within the context of the present invention, the term “ingredient providing nutrient X” or “ingredients providing nutrient X” indicates an edible substance and/or mixture of substances which comprise or is essentially consisting of at least one substance capable of delivering the specified nutrient X to the human body.

Within the context of the present invention, the term “ingredient providing nutrient X in amount Y” or “ingredients providing nutrient X in amount Y” indicates an edible substance and/or mixture of substances which comprise or is essentially consisting of at least one substance capable of delivering the specified nutrient X to the human body in the specified amount Y.

Insoluble fiber does not dissolve in water, is metabolically inert and provides bulking, or it can be prebiotic and metabolically ferment in the large intestine. Chemically, dietary fiber consists of non-starch polysaccharides (NPS) such as arabinoxylans, cellulose, and many other plant components such as restistant oligosaccharides, resistant starch, resistant dextrins, inulin, lignin, chitins, pectins, beta-glucans, and oligosaccharides. Non limiting examples of dietary fibers are: prebiotic fibers such as Fructo-oligosaccharides (FOS), inulin, galacto-oligosaccharides (GOS), fruit fiber, legume fiber, vegetable fiber, cereal fiber, resistant starch such as high amylose corn starch. As fibers are not digestible, they do not contain available carbohydrates.

The expressions “fiber” or “fibers” or “dietary fiber” or “dietary fibers” within the context of the present invention indicate the indigestible portion, in small intestine, of food derived from plants which comprises two main components: Soluble fiber, which dissolves in water, and insoluble fiber. Mixtures of fibers are comprised within the scope of the terms above mentioned. Soluble fiber is readily fermented in the colon into gases and physiologically active byproducts, and can be prebiotic and viscous.

Within the context of the present invention the term “added fiber” or “added dietary fiber” indicates an ingredient mainly or totally constituted by fiber which is added to the nutritional composition according to the present invention and whose content in fiber contributes to the total fiber content of the composition. The total fiber content of the nutritional composition is provided by the sum of amount of fiber naturally present in ingredients used in the recipe (for example from whole grain cereal flour) plus amount of added fiber.

Within the context of the present invention, the term “legume” or “legumes” identifies the fruit or seed of a plant in the family of Fabaceae or mixtures thereof. Well-known legumes include inter alia alfalfa, clover, peas, beans, lentils, lupins, mesquite, carob, soybeans, peanuts and tamarind. The grain seeds of such plants are generally known as “pulses” and are comprised within the scope of the term “legumes” according to the present invention.

Within the context of the present invention, the term “fruit” or “fruits” indicates ingredients derived from fruit such as for example fresh fruit, fruit paste, dried fruit, fruit extracts and/or centrifugates. Mixtures of such ingredients are also comprised within the scope of the terms above mentioned. Non limiting examples of fruit according to the present invention are: apple, apricot, banana, cherry, pear, strawberry, Mango, Orange, peach.

As it will be apparent to the skilled person, legumes and fruit according to the present invention may bring certain amount of fibers to the nutritional composition of the present invention.

The composition of the present invention, including the many embodiments described herein, can comprise, consist of, or consist essentially of the essential elements and limitations of the invention described herein, as well as any additional or optional ingredients, components, or limitations described herein.

### Embodiments of the Invention

In one embodiment, the mastitis is sub-clinical mastitis or clinical mastitis.

In a preferred embodiment, the mastitis is sub-clinical mastitis.

In one embodiment, the subject is at risk of suffering from sub-clinical mastitis or clinical mastitis.

In one embodiment, the risk of suffering from mastitis (such as sub-clinical mastitis or clinical mastitis) is indicated by the presence of one or more risk factors selected from the group consisting of family history of sub-clinical mastitis or clinical mastitis, breast-feeding attachment difficulties, mother-infant separation (e.g. separation of greater than 24 h), blocked duct, milk stasis, cracked nipples, pre-lacteal feeds, milk oversupply, breast engorgement, feeding from alternate breasts on consecutive feeds, infant mouth abnormalities, a short infant frenulum, maternal use of antibiotics, previous history of mastitis in the subject, maternal stress, delivery in private versus public hospital and the presence of Staphylococcus aureus in milk.

In one embodiment, the subject is a human e.g. a woman who is desiring to get pregnant, who is pregnant or who is lactating.

In one embodiment, the subject is a human e.g. a lactating woman. In another embodiment, the subject is a lactating woman.

In one embodiment, the subject is a livestock animal or a companion animal. In one embodiment, the subject is a cow or dog. In another embodiment, the subject is a rat or mouse.

In one embodiment, the treatment or prevention increases the probability of initiating and/or continuing breastfeeding by the subject.

In one embodiment, the treatment or prevention increases the probability of the subject exclusively breast-feeding her infant.

In one embodiment, the treatment or prevention increases the duration (length of time e.g. number of days, weeks, months) of breastfeeding by the subject.

In one embodiment, the subject is able to breast-feed for at least 4 months, preferably 4-24 months, optionally 4-6 months.

In one embodiment, the subject is able to breast-feed for at least 6 months, preferably 6-24 months.

In one embodiment, the treatment or prevention increases the quality of the subject’s breast milk.

In one embodiment, the treatment or preventing increases the quantity of the subject’s breast milk.

### Mastitis

Mastitis is an inflammation of the mammary gland tissue, which can be classified as sub-clinical or clinical depending on the degree of inflammation.

Clinical mastitis is a form of mastitis associated with reduced milk secretion, visible signs of inflammation of the breast and, changes in the appearance of milk, which may be accompanied by systemic signs. Sub-clinical mastitis is a form of mastitis characterised by reduced milk secretion and a high milk bacterial count in the absence of evident inflammatory changes, including pain (Fernandez, L. et al. (2014) Beneficial Microbes 5: 169-183).

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain.

Staphylococcus infections, in particular S. aureus and S. epidermidis infections, are understood to be a primary cause of mastitis.

Mastitis can result in curtailment or even lack of initiation of breast-feeding of an infant. Furthermore, the composition of breast milk may change during mastitis, for example increasing in content of sodium and inflammatory mediators, which may adversely affect the nutrition provided to the infant.

In one embodiment of the present invention, mastitis is sub-clinical mastitis.

### Method for Assessing the Risk of Developing Mastitis

In one aspect, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) based on the dietary intake data of step a);
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

In an additional aspect, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) based on the dietary intake data of step a);
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.
wherein the method is computer implemented.

In one embodiment, the method according to the present invention can be carried out by computer program instructions running on means which provide generic data processing function. Such means can be embedded, for example in a personal computer, a smartphone, a tablet, etc.

In one embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) on the basis of dietary intake data of step a) by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, for example a lactating woman;
c) associating to such overall Dietary Inflammatory Index score a level of risk of developing mastitis, for example subclinical mastitis, for the subject; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

In a further embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) based on the dietary intake data of step a);
c) associating to such overall Dietary Inflammatory Index score a level of risk for the subject of developing mastitis, for example subclinical mastitis, which is:
   low risk if the subject has an overall Dietary Inflammatory Index score lower than 0.8;
   medium risk if the subject has an overall Dietary Inflammatory Index score comprised between 0.8 and 1.68;
   high risk if the subject has an overall Dietary Inflammatory Index score higher than 1.68; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

In a still further embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) on the basis of dietary intake data of step a) by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, for example a lactating woman;
c) associating to such overall Dietary Inflammatory Index score a level of risk for the subject of developing mastitis, for example subclinical mastitis, which is:
   low risk if the subject has an overall Dietary Inflammatory Index score lower than 0.8;
   medium risk if the subject has an overall Dietary Inflammatory Index score comprised between 0.8 and 1.68;
   high risk if the subject has an overall Dietary Inflammatory Index score higher than 1.68; and optionally
d) recommending to the subject to follow a diet with an overall Dietary inflammatory index score lower than 0.8 to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

In another embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) on the basis of dietary intake data of step a) by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, for example a lactating woman;
c) associating to such overall Dietary Inflammatory Index score a level of risk for the subject of developing mastitis, for example subclinical mastitis, which is:
   low risk if the subject has an overall Dietary Inflammatory Index score lower than 0.8;
   medium risk if the subject has an overall Dietary Inflammatory Index score comprised between 0.8 and 1.68;
   high risk if the subject has an overall Dietary Inflammatory Index score higher than 1.68;
d) if risk for the subject is medium or high, recommending to the subject:
   to follow a diet with an overall Dietary inflammatory index score lower than 0.8; and/or
   to consume food parameters, or compositions comprising them, having a food parameter-specific overall inflammatory effect score lower than 0; and /or
   to consume food parameters, or composition comprising them, which are selected in the list consisting of: potassium, Vitamin B5, Vitamin B12, Manganese, copper, calcium, phosphorus, iron, phosphatidyl choline, alphalactalbumin, lactoferrin, albumin, w-3 fatty acids, especially DHA and/or EPA, and combinations thereof; and/or
   to limit the consumption of food parameters, or compositions comprising them, having a food parameter-specific overall inflammatory effect score higher than 0.

In another embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) on the basis of dietary intake data of step a) by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, for example a lactating woman;
c) associating to such overall Dietary Inflammatory Index score a level of risk for the subject of developing mastitis, for example subclinical mastitis, which is:
   low risk if the subject has an overall Dietary Inflammatory Index score lower than 0.8;
   medium risk if the subject has an overall Dietary Inflammatory Index score comprised between 0.8 and 1.68;
   high risk if the subject has an overall Dietary Inflammatory Index score higher than 1.68;
d) if risk for the subject is medium or high, recommending to the subject:
   to follow a diet with an overall Dietary inflammatory index score lower than 0.8;and/or
   to consume food parameters, or compositions comprising them, having a food parameter-specific overall inflammatory effect score lower than 0, which are selected in the group consisting of: alcohol, Vitamin B6, beta-carotene, caffeine, carbohydrates, fibers, folic acid, garlic, ginger, magnesium, n-3 fatty acids, n-6 fatty acids, onion, Polyunsaturated fatty acids, Vitamin B2, saffron, Selenium, Vitamin B1, turmeric, Vitamin A, Vitamin C, Vitamin D, Vitamin E, Zinc, green and/r black tea, flavan-3-ol, flavones, flavonols, flavonones, antocyanidins, isoflavones, pepper, thyme and/or oregano, rosemary and combinations thereof; and /or
   to consume food parameters, or composition comprising them, which are selected in the list consisting of: potassium, Vitamin B12, Vitamin B5, Manganese, copper, calcium, phosphorus, iron, phosphatidylcholine , alphalactalbumin, lactoferrin, albumin, n-3 fatty acids, especially DHA and/or EPA, and combinations thereof; and/or
   to limit the consumption of food parameters, or compositions comprising them, having a food parameter-specific overall inflammatory effect score higher than 0, such as saturated fats, for example selected in the group consisting of: butyric acid, caproic acid, caprylic acid, capric acid, mystiric acid, palmitic acid, stearic acid and combinations thereof.

In a further additional embodiment, the present invention provides for a method to assess the risk of developing mastitis, for example subclinical mastitis, in a subject, for example a lactating woman, the method comprising:
a) recording dietary intake data of the subject, for example recording dietary intake of food parameters having a food parameter-specific overall inflammatory effect score;
b) calculate the overall Dietary Inflammatory Index score with the procedure described in Shivappa et al, Public Health Nutrition, 17 (8), 1689-1696) on the basis of dietary intake data of step a) by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, for example a lactating woman;
c) associating to such overall Dietary Inflammatory Index score a level of risk for the subject of developing mastitis, for example subclinical mastitis, which is:
   low risk if the subject has an overall Dietary Inflammatory Index score lower than 0.8;
   medium risk if the subject has an overall Dietary Inflammatory Index score comprised between 0.8 and 1.68;
   high risk if the subject has an overall Dietary Inflammatory Index score higher than 1.68;
d) if risk for the subject is medium or high, recommending to the subject:
   to follow a diet with an overall Dietary inflammatory index score lower than 0.8;and/or
   to consume food parameters, or compositions comprising them, having a food parameter-specific overall inflammatory effect score lower than 0, which are selected in the group consisting of: Vitamin B6, beta-carotene, fibers, folic acid, magnesium, n-3 fatty acids (such as for example DHA, EPA and/or ALA), n-6 fatty acids (such as for example linoleic acid), Vitamin B2, Selenium, Vitamin B1, Vitamin A, Vitamin C, Vitamin D, Vitamin E, Zinc, and combinations thereof; and /or
   to consume food parameters, or composition comprising them, which are selected in the list consisting of: potassium, Vitamin B12, Vitamin B5, Manganese, copper, calcium, phosphorus, iron, phosphatidylcholine, alphalactalbumin, lactoferrin, albumin and combinations thereof; and /or
   to limit the consumption of food parameters, or compositions comprising them, having a food parameter-specific overall inflammatory effect score higher than 0, such as saturated fats, for example selected in the group consisting of: butyric acid, caproic acid, caprylic acid, capric acid, mystiric acid, palmitic acid, stearic acid and combinations thereof.

### Methods of Treatment

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

It is to be appreciated that all references herein to treatment include curative, palliative and prophylactic treatment. The treatment of mammals, particularly humans, is preferred. Both human and veterinary treatments are within the scope of the invention.

The minerals, fatty acids, proteins, combinations and compositions disclosed herein may be administered to a woman desiring to get pregnant, to a pregnant woman and/or to a lactating woman.

If administration is to a pregnant woman, administration may be for example for at least 4, at least 8, at least 12, at least 16, at least 20, at least 24, at least 28 or at least 36 weeks during pregnancy. As the nutritional requirements increase in the second and third trimester of pregnancy, it may be particularly beneficial if administration is throughout the second and/or third trimester of pregnancy.

Administration pre-pregnancy and/or during pregnancy may enable a woman to build up a store of one or more of the nutrients before lactation.

If administration is to a lactating woman, administration may be for example for any part of the lactation period for example up to 2 years, up to 1 year, up to 9, 8, 7, 6, 5, 4, 3, 2 or 1 months post birth.

In one embodiment, administration is to a woman desiring to get pregnant, to a pregnant woman and/or to a lactating woman.

### Composition

In one embodiment, the composition is a nutritional composition or a pharmaceutical composition, preferably a nutritional composition.

In one embodiment, the composition is a maternal nutritional composition, preferably for use during lactation and/or pregnancy, for example late pregnancy. In another embodiment, the composition is a maternal nutritional composition for use during lactation.

In one embodiment, the nutrient or composition for use according to the present invention is in the form of a tablet, gel capsule, powder, maternal milk powder, food product, liquid format (e.g. ready to drink format) and/or beverage.

The term “maternal nutritional composition” as used herein refers to any composition that has been specifically manufactured for consumption by a pregnant woman, a woman trying to conceive or a lactating woman, or a composition that is specifically marketed at pregnant women, women trying to conceive or lactating (e.g. breast-feeding) women.

The maternal nutritional composition may be, for example, a food product, a functional food product, a drink (beverage), a dairy product or dairy substitute product, a pharmaceutical formulation or a supplement.

The term “dairy product” as used herein refers to food products produced from animals such as cows, goats, sheep, yaks, horses, camels and other mammals. Examples of dairy products are low-fat milk (e.g. 0.1%, 0.5% or 1.5% fat milk), fat-free milk, milk powder, whole milk, whole milk products, butter, buttermilk, buttermilk products, skim milk, lactose-free products, high milk-fat products, condensed milk, crème fraiche, cheese, ice cream and confectionery products, probiotic drinks or probiotic yoghurt-type drinks. A dairy substitute product may be a soya, almond or vegetable-based dairy substitute, e.g. a milk or yoghurt substitute.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “beverage product” as used herein refers to a nutritional product in liquid or semiliquid form that may be safely consumed by an individual. The beverage product may be a water-based product, such as a product in which the agents of the invention are dissolved or suspended in water.

The term “food product” as used herein refers to any kind of product that may be safely consumed by a woman, in particular a pregnant woman, a woman trying to conceive or a lactating (e.g. breast-feeding) woman. Said food product may be in solid, semi-solid or liquid form and may comprise one or more nutrients, foods or nutritional supplements. For example, the food product may further comprise one or more of the following nutrients and micronutrients: a source of protein, a source of lipid, a source of carbohydrate, vitamins and minerals. The composition may also contain anti-oxidants, stabilisers (when provided in solid form) or emulsifiers (when provided in liquid form).

The term “functional food product” as used herein refers to a food product providing an additional health-promoting or disease-preventing function to the individual. Food products and functional food products include, for example, cereal-based products, yoghurts or other milk-derived products and bars.

The term “supplement” as used herein refers to a nutritional product that provides nutrients (e.g. vitamins and/or minerals) to an individual that may otherwise not be consumed in sufficient quantities by said individual. Supplements may be, for example, provided in the form of a pill, a tablet, a lozenge, a chewy capsule or tablet, a capsule, or a powder supplement that can be, for example, dissolved in water or milk, or sprinkled on food. Supplements typically provide selected nutrients without providing a significant portion of the overall nutritional needs of a subject. Typically supplements do not represent more than 0.1%, 1%, 5%, 10% or 20% of the daily energy need of a subject. In the context of the present invention the subject may be, for example, a woman trying to get pregnant, a pregnant woman and/or a lactating woman.

The term “pregnancy supplement” as used herein refers to a supplement that is specifically formulated for administration to a woman who is trying to conceive and/or to a woman who is pregnant, or marketed towards a woman who is trying to conceive and/or a woman who is pregnant.

The term “lactation supplement” as used herein refers to a supplement that is specifically formulated for administration to a woman who is lactating, or marketed toward a woman who is lactating. Consumption of lactation supplements may be advised to commence during pregnancy.The compositions of the invention may also comprise ingredients commonly used in maternal nutritional compositions. Non-limiting examples of such ingredients include: probiotics, lipids, carbohydrates, pharmaceutically-active agents and conventional additives, such as anti-oxidants, stabilisers, emulsifiers, acidulants, thickeners, buffers or agents for pH adjustment, chelating agents, colourants, excipients, flavour agents, osmotic agents, pharmaceutically-acceptable carriers, preservatives, sugars, sweeteners, texturisers, emulsifiers and water.

It may also be beneficial if the compositions of the invention comprise probiotics. Probiotics may help nutrients pass through the gut.

The term “probiotic” as used herein refers to live probiotic bacteria, non-replicating probiotic bacteria, dead probiotic bacteria, non-viable probiotic bacteria, fragments of probiotic bacteria, such as DNA, metabolites of probiotic bacteria, cytoplasmic compounds of probiotic bacteria, cell wall materials of probiotic bacteria, culture supernatants of probiotic bacteria, and combinations of any of the foregoing.

The probiotic may be live probiotic bacteria, non-replicating probiotic bacteria, dead probiotic bacteria, non-viable probiotic bacteria and any combination thereof.

### Composition Comprising Food Parameters

In a further aspect of the present invention, a nutritional composition is provided comprising at least one food parameter having a food parameter-specific overall inflammatory effect score lower than 0 for the prevention of treatment of mastitis, for example subclinical mastitis.

In one embodiment, such nutritional composition comprises at least a food parameter selected in the group consisting of: Vitamin B6, beta-carotene, fibers, folic acid, magnesium, n-3 fatty acids (such as for example DHA, EPA and/or ALA), n-6 fatty acids (such as for example linoleic acid), Vitamin B2, Selenium, Vitamin B1, Vitamin A, Vitamin C, Vitamin D, Vitamin E, Zinc, and combinations thereof.

In another embodiment, a nutritional composition is provided comprising at least a food parameter selected in the group consisting of:: Vitamin B6, beta-carotene, fibers, folic acid, magnesium, n-3 fatty acids (such as for example DHA, EPA and/or ALA), n-6 fatty acids (such as for example linoleic acid), Vitamin B2, Selenium, Vitamin B1, Vitamin A, Vitamin C, Vitamin D, Vitamin E, Zinc, and combinations thereof; and comprising at least a food parameter selected in the list consisting of: potassium, Vitamin B12, Vitamin B5, Manganese, copper, calcium, phosphorus, iron, phosphatidyl choline, alphalactalbumin, lactoferrin, albumin and combinations thereof.

### Subject

The term “subject” as used herein refers to either a human or non-human animal. The non-human animal may be, for example, a livestock animal or a companion animal.

A “companion animal” is any domesticated animal, and includes, without limitation, cats, dogs, rabbits, guinea pigs, ferrets, hamsters, mice, gerbils, horses, cows, goats, sheep, donkeys, pigs and the like.

In one embodiment, the subject is a human subject. In another embodiment, the subject is a companion animal. Preferably, the subject is a human.

In one embodiment, the subject is at risk of mastitis and/or subclinical mastitis. In another embodiment, the subject is a lactating animal.

In one embodiment, the human subject is a woman. In a further embodiment, the human subject is a lactating woman. In another embodiment, the human subject is a pregnant woman.

In one embodiment, the human subject is a European and or Caucasian woman. In a further embodiment, the human subject is a European and or Caucasian lactating woman. In another embodiment, the human subject is a European and or Caucasian pregnant woman.

In a still further embodiment, the human subject is a woman at risk of mastitis and/or of subclinical mastitis.

In another embodiment, the human subject is a lactating woman at risk of mastitis and/or of subclinical mastitis.

### Treating and Preventing

The term “prevent” as used herein includes prevention and reducing the risk of a condition.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature.

### Food Parameters

### Fiber

In one embodiment of the present invention, a composition is provided which comprises at least one ingredient which delivers fibres.

In one embodiment of the present invention, a nutritional composition is provided which comprises fibres.

In one embodiment, the ingredients providing fibres may be capable of providing fibres of natural or synthetic origin.

In one embodiment, fibres of synthetic origin are for example FOS from sucrose.

In one embodiment, ingredients which are capable of providing dietary fibres are selected in the group consisting of: Fruit, Vegetable, Legume, Cereal and Cruciferous vegetable.

In one embodiment, dietary fibres are selected in the group consisting of: resistant dextrin, resistant oligosaccharides, NPS, resistant starches (for example pectine), polydextrose, inulin, partially hydrolyzed guar gum (PHGG), FOS, acacia gum, pea fiber, and mixtures thereof.

As it is evident to the person skilled in the art, different ingredients may provide different amounts of dietary fibres in the composition according to the present invention, depending on the nature and amount of the ingredient used. It will be nonetheless routine work to the skilled person to calculate the amount of ingredient needed to provide the claimed amount of dietary fibres, based on the specification of the specific ingredient provided by the supplier.

In one embodiment, the dosage of fibers is at least 17 g/day. In a further embodiment, the dosage of fibers is at least 20 g/day. In a still further embodiment, the dosage of fibers is ranging from 16 to 30 g/day, for example 20 to 25 g/day.

In one embodiment, the dosage of fibers as above described is adapted for a lactating woman.

The fibers may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of fibers as above described. In such embodiment, as it will be apparent to a person skilled in the art, the amount of fibers contained in each serving of the composition according to the present invention will be divided by 1, 2 3 or 4 respectively.

In one embodiment, the composition according to the present invention is intended for consumption once or twice per day.

### Beta-Carotene

Beta-carotene may be incorporated in the composition of the invention as such or via any source comprising it. For example ingredients providing beta-carotene may be synthetic or natural sources.

As it is evident to the person skilled in the art, different ingredients may provide different amounts of beta-carotene in the composition according to the present invention, depending on the nature and amount of the ingredient used. It will be nonetheless routine work to the skilled person to calculate the amount of ingredient needed to provide the claimed amount of beta-carotene, based on the specification of the specific ingredient provided by the supplier.

In one embodiment, the dosage of beta-carotene is at least 2100 µg/day. In a further embodiment, the dosage of beta-carotene is at least 2400 µg/day. In a still further embodiment, the dosage of beta-carotene is ranging from 2100 to 3500 µg/day, for example from 2400 to 3500 µg/day.

In such embodiment, the composition of the present invention delivers the daily amount of beta-carotene considered responsible for the beneficial observed effect on occurrence of mastitis, especially subclinical mastitis.

### Vitamin C

Vitamin C may be incorporated in the composition of the invention as such or in the form of a physiologically acceptable salt and/or via any source comprising Vitamin C. For example ingredients may be selected in the group consisting of: ascorbic acid, sodium ascorbate and mixtures thereof.

As it is evident to the person skilled in the art, different ingredients may provide different amounts of Vitamin C in the composition according to the present invention, depending on the nature and amount of the ingredient used. It will be nonetheless routine work to the skilled person to calculate the amount of ingredient needed to provide the claimed amount of Vitamin C, based on the specification of the specific ingredient provided by the supplier.

In one embodiment, the dosage of Vitamin C is at least 100 mg/day. In a further embodiment, the dosage of Vitamin C is at least 105 mg/day. In a still further embodiment, the dosage of Vitamin C is ranging from 100 to 2000 mg/day, for example from 105 to 200 mg/day.

In such embodiment, the composition of the present invention delivers the daily amount of Vitamin C considered responsible for the beneficial observed effect on occurrence of mastitis, especially subclinical mastitis.

In one embodiment, the dosage of vitamin C as above described is adapted for a lactating woman.

The vitamin C may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin C as above described. In such embodiment, as it will be apparent to a person skilled in the art, the amount of Vitamin C contained in each serving of the composition according to the present invention will be divided by 1, 2 3 or 4 respectively.

In one embodiment, the composition according to the present invention is intended for consumption once or twice per day.

### Folate

Folate may be incorporated in the nutritional compositions of the invention as folic acid or in the form of a physiologically acceptable salt thereof (folate) or mixtures thereof.

In one embodiment, folate is of synthetic origin.

As it is evident to the person skilled in the art, different ingredients may provide different amounts of folate in the context of the present invention, depending on the nature and amount of the ingredient used. It will be nonetheless routine work to the skilled person to calculate the amount of ingredient needed to provide the claimed amount of folate, based on the specification of the specific ingredient provided by the supplier.

In one embodiment, the dosage of folate is at least 305 µg/day. In a further embodiment, the dosage of folate is at least 310 µg/day. In a still further embodiment, the dosage of folate is ranging from 305 to 1000 µg/day, for example from 310 to 600 µg/day.

In such embodiment, the composition of the present invention delivers the daily amount of folate considered responsible for the beneficial observed effect on occurrence of mastitis, especially subclinical mastitis.

The folate may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of folate as above described. In such embodiment, as it will be apparent to a person skilled in the art, the amount of folate contained in each serving of the composition according to the present invention will be divided by 1, 2 3 or 4 respectively.

In one embodiment, the composition according to the present invention is intended for consumption once or twice per day.

### Potassium

Potassium may be provided in the context of the present invention as such or in the form of a physiologically acceptable salt and/or via any source comprising Potassium. For example ingredients may be selected in the group consisting of: potassium phosphate, potassium sulfate, potassium citrate, Potassium chloride, potassium aspartate, potassium bicarbonate, potassium gluconate and mixtures thereof.

In one embodiment, potassium is provided by potassium chloride.

As it is evident to the person skilled in the art, different ingredients may provide different amounts of potassium in the context of the present invention, depending on the nature and amount of the ingredient used. It will be nonetheless routine work to the skilled person to calculate the amount of ingredient needed to provide the claimed amount of potassium, based on the specification of the specific ingredient provided by the supplier.

In one embodiment, the dosage of potassium is at least 2800 mg/day. In a further embodiment, the dosage of potassium is at least 3000 mg/day. In a still further embodiment, the dosage of potassium is ranging from 2800 to 5000 mg/day, for example from 3000 to 4000 mg/day.

In such embodiment, the composition of the present invention delivers the daily amount of Potassium considered responsible for the beneficial observed effect on occurrence of mastitis, especially subclinical mastitis.

In one embodiment, the dosage of potassium as above described is adapted for a lactating woman.

The potassium may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Potassium as above described. In such embodiment, as it will be apparent to a person skilled in the art, the amount of Potassium contained in each serving of the composition according to the present invention will be divided by 1, 2 3 or 4 respectively.

In one embodiment, the composition according to the present invention is intended for consumption once or twice per day.

### Vitamin B12 (Cobalamin)

Vitamin B12 may be incorporated in the composition of the invention as such or in the form of a physiologically acceptable salt and/or via any source comprising Vitamin B12. For example ingredients may be selected in the group consisting of: Cyanocobalamin, methylcobalamin, adenosylcobalamin and hydroxocobalamin.

As it is evident to the person skilled in the art, different ingredients may provide different amounts of Vitamin B12 in the composition according to the present invention, depending on the nature and amount of the ingredient used. It will be nonetheless routine work to the skilled person to calculate the amount of ingredient needed to provide the claimed amount of Vitamin B12, based on the specification of the specific ingredient provided by the supplier.

In one embodiment, the dosage of Vitamin B12 is at least 5 µg/day. In a further embodiment, the dosage of Vitamin B12 is at least 5.5 µg/day. In a still further embodiment, the dosage of Vitamin B12 is ranging from 5 to 250 µg/day, for example from 5.5 to 100 µg/day.

In such embodiment, the composition of the present invention delivers the daily amount of Vitamin B12 considered responsible for the beneficial observed effect on occurrence of mastitis, especially subclinical mastitis.

In one embodiment, the dosage of vitamin B12 as above described is adapted for a lactating woman.

The vitamin B12 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B12 as above described. In such embodiment, as it will be apparent to a person skilled in the art, the amount of Vitamin B12 contained in each serving of the composition according to the present invention will be divided by 1, 2 3 or 4 respectively.

In one embodiment, the composition according to the present invention is intended for consumption once or twice per day.

### Vitamin B6

Vitamin B16 may be incorporated in the composition of the invention as such or in the form of a physiologically acceptable salt and/or via any source comprising Vitamin B6. For example ingredients may be selected in the group consisting of: pyridoxine (in the form of pyridoxine hydrochloride [HCI]) and pyridoxal 5' phosphate (PLP).

As it is evident to the person skilled in the art, different ingredients may provide different amounts of Vitamin B6 in the composition according to the present invention, depending on the nature and amount of the ingredient used. It will be nonetheless routine work to the skilled person to calculate the amount of ingredient needed to provide the claimed amount of Vitamin B6, based on the specification of the specific ingredient provided by the supplier.

In one embodiment, the dosage of Vitamin B6 is at least 1.9 mg day. In a further embodiment, the dosage of Vitamin B6 is at least 2.0 mg/day. In a still further embodiment, the dosage of Vitamin B6 is ranging from 1.9 to 100 mg/day, for example from 2.0 to 50 mg/day.

In such embodiment, the composition of the present invention delivers the daily amount of Vitamin B6 considered responsible for the beneficial observed effect on occurrence of mastitis, especially subclinical mastitis.

In one embodiment, the dosage of vitamin B6 as above described is adapted for a lactating woman.

The vitamin B6 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B6 as above described. In such embodiment, as it will be apparent to a person skilled in the art, the amount of Vitamin B6 contained in each serving of the composition according to the present invention will be divided by 1, 2 3 or 4 respectively.

In one embodiment, the composition according to the present invention is intended for consumption once or twice per day.

### Vitamin B5 (Pantothenic Acid)

Vitamin B5 may be incorporated in the composition of the invention as such or in the form of a physiologically acceptable salt and/or via any source comprising Vitamin B5. For example ingredients may be selected in the group consisting of: Pantothenic acid, pantethine, pantetheine and calcium pantothenate.

As it is evident to the person skilled in the art, different ingredients may provide different amounts of Vitamin B5 in the composition according to the present invention, depending on the nature and amount of the ingredient used. It will be nonetheless routine work to the skilled person to calculate the amount of ingredient needed to provide the claimed amount of Vitamin B5, based on the specification of the specific ingredient provided by the supplier.

In one embodiment, the dosage of Vitamin B5 is at least 5.3 mg/day. In a further embodiment, the dosage of Vitamin B5 is at least 5.5 mg/day. In a still further embodiment, the dosage of Vitamin C is ranging from 5.3 to 1500 mg/day, for example from 5.5 to 200 mg/day.

In such embodiment, the composition of the present invention delivers the daily amount of Vitamin B5 considered responsible for the beneficial observed effect on occurrence of mastitis, especially subclinical mastitis.

In one embodiment, the dosage of vitamin B5 as above described is adapted for a lactating woman.

The vitamin B5 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B5 as above described. In such embodiment, as it will be apparent to a person skilled in the art, the amount of Vitamin B5 contained in each serving of the composition according to the present invention will be divided by 1, 2 3 or 4 respectively.

In one embodiment, the composition according to the present invention is intended for consumption once or twice per day.

### Vitamin B2 (Riboflavin)

Vitamin B2 may be incorporated in the composition of the invention as such or in the form of a physiologically acceptable salt and/or via any source comprising Vitamin B2. For example ingredients may be selected in the group consisting of: riboflavin and riboflavin 5'-monophosphate.

As it is evident to the person skilled in the art, different ingredients may provide different amounts of Vitamin B2 in the composition according to the present invention, depending on the nature and amount of the ingredient used. It will be nonetheless routine work to the skilled person to calculate the amount of ingredient needed to provide the claimed amount of Vitamin B2, based on the specification of the specific ingredient provided by the supplier.

In one embodiment, the dosage of Vitamin B2 is at least 1.8 mg/day. In a further embodiment, the dosage of Vitamin B2 is at least 2.0 mg/day. In a still further embodiment, the dosage of Vitamin B2 is ranging from 1.8 to 5 mg/day, for example from 2.0 to 4 mg/day.

In such embodiment, the composition of the present invention delivers the daily amount of Vitamin B2 considered responsible for the beneficial observed effect on occurrence of mastitis, especially subclinical mastitis.

In one embodiment, the dosage of vitamin B2 as above described is adapted for a lactating woman.

The vitamin B2 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B2 as above described. In such embodiment, as it will be apparent to a person skilled in the art, the amount of Vitamin B2 contained in each serving of the composition according to the present invention will be divided by 1, 2 3 or 4 respectively.

In one embodiment, the composition according to the present invention is intended for consumption once or twice per day.

### Vitamin B1 (Thiamin)

Vitamin B1 may be incorporated in the composition of the invention as such or in the form of a physiologically acceptable salt and/or via any source comprising Vitamin B1. For example ingredients may be selected in the group consisting of: thiamin mononitrate and thiamin hydrochloride.

As it is evident to the person skilled in the art, different ingredients may provide different amounts of Vitamin B1 in the composition according to the present invention, depending on the nature and amount of the ingredient used. It will be nonetheless routine work to the skilled person to calculate the amount of ingredient needed to provide the claimed amount of Vitamin B1, based on the specification of the specific ingredient provided by the supplier.

In one embodiment, the dosage of Vitamin B1 is at least 1.6 mg/day. In a further embodiment, the dosage of Vitamin B1 is at least 1.8 mg/day. In a still further embodiment, the dosage of Vitamin B1 is ranging from 1.6 to 500 mg/day, for example from 1.8 to 5 mg/day.

In such embodiment, the composition of the present invention delivers the daily amount of Vitamin B1 considered responsible for the beneficial observed effect on occurrence of mastitis, especially subclinical mastitis.

The vitamin B1 may be comprised in any form suitable for ingestion by a woman such as a pregnant woman, a woman trying to conceive or a lactating woman.

In one embodiment, the composition according to the present invention may be administered in 1, 2, 3 or 4 daily servings to provide the total daily amounts of Vitamin B1 as above described. In such embodiment, as it will be apparent to a person skilled in the art, the amount of Vitamin B1 contained in each serving of the composition according to the present invention will be divided by 1, 2 3 or 4 respectively.

In one embodiment, the composition according to the present invention is intended for consumption once or twice per day.

The minerals may be used in any form suitable for ingestion by animals, preferably humans (e.g. are non-toxic). The minerals may be used, for example in compositions such as nutritional compositions, in any appropriate amount. The skilled person will be able tod determine appropriate amounts depending on the desired dosage of the mineral. Dosages may depend on factors such as the age, size and health status of the woman to whom they are administered, on her lifestyle, as well as on her genetic heritage. Dosages may be in line with the recommended daily allowance (RDA) developed by organisations such as the Food and Nutrition Board of the National Academy of Sciences.

The skilled person can readily determine an appropriate dose of one of the agents of the invention to administer to a subject without undue experimentation. Typically, a physician will determine the actual dosage that will be most suitable for an individual subject and it will depend on a variety of factors including the activity of the specific agent employed, the metabolic stability and length of action of that agent, the age, body weight, general health, sex, diet, mode and time of administration, rate of excretion, drug combination, the severity of the particular condition and the individual undergoing therapy. There can of course be individual instances where higher or lower dosage ranges are merited.

### Iron

In one embodiment, the dosage of iron is about 2.7-45, 5-25 or 9-10 mg/day. A dosage of about 9-10 mg/day may be preferred for breast-feeding women.

In another embodiment, the dosage of iron is about 30-60 mg/day. A dosage of about 30-60 mg/day may be preferred for pregnant women.

In one embodiment, the dosage of iron is at least 9.1 m/dayg. In a further embodiment, the dosage of iron is at least 9.5 mg/day. In a still further embodiment, the dosage of iron is ranging from 9.5 to 60 mg/day, for example from 9.5 to 50 mg/day/day, for example 9.5 to 40 mg/day.

In one embodiment, the dosage of iron for a lactating woman is at least 9.1 mg/day. In a further embodiment, the dosage of iron is at least 9.5 mg/day. In a still further embodiment, the dosage of iron is ranging from 9.5 to 60 mg/day, for example from 9.5 to 30 mg/day, for example 9.5 to 20 mg/day.

In one embodiment, the dosage of iron is at least 11.6 mg/day. In a further embodiment, the dosage of iron is at least 12 mg/day. In a still further embodiment, the dosage of iron is ranging from 12 to 60 mg/day, for example from 12 to 50 mg/day, for example 12 to 40 mg/day.

In one embodiment, the dosage of iron for a lactating woman is at least 11.6 mg/day. In a further embodiment, the dosage of iron is at least 12 mg/day. In a still further embodiment, the dosage of iron is ranging from 12 to 60 mg/day, for example from 12 to 30 mg/day, for example 12 to 20 mg/day.

The iron may be comprised in any form suitable for ingestion by a woman such as a pregnant woman, a woman trying to conceive or a lactating woman. For example, iron may be comprised in the form of iron sulfate, iron citrate, iron choline citrate, iron ammonium citrate, iron chloride, iron fumarate, iron gluconate, iron pyroposphate or a mixture thereof.

### Manganese

In one embodiment, the dosage of manganese is about 1.8-11, 2-3 or 2.5-2.7 mg/day. A dosage of about 2.5-2.7 mg/day may be preferred for breast-feeding women. A dosage of about 1.9-2.1 mg/day may be preferred for pregnant women.

In one embodiment, the dosage of manganese is at least 2.1 mg/day. In a further embodiment, the dosage of manganese is at least 2.3 mg/day. In a still further embodiment, the dosage of managese is ranging from 2.1 to 4 mg/day, for example from 2.3 to 3.5 mg/day.

In one embodiment, the dosage of manganese for a lactating woman is at least 2.1 mg/day. In a further embodiment, the dosage of manganese is at least 2.3 mg/day. In a still further embodiment, the dosage of managese is ranging from 2.1 to 4 mg/day, for example from 2.3 to 3.5 mg/day.

In one embodiment, the dosage of manganese is at least 2.6 mg/day. In a further embodiment, the dosage of manganese is at least 3.0 mg/day. In a still further embodiment, the dosage of managese is ranging from 2.6 to 4 mg/day, for example from 3.0 to 3.5 mg/day.

In one embodiment, the dosage of manganese for a lactating woman is at least 2.6 mg/day. In a further embodiment, the dosage of manganese is at least 3.0 mg/day. In a still further embodiment, the dosage of managese is ranging from 2.6 to 4 mg/day, for example from 3.0 to 3.5 mg/day.

The manganese may be comprised in any form suitable for ingestion by a woman such as a pregnant woman, a woman trying to conceive or a lactating woman. For example, manganese may be comprised in the form of manganese gluconate, manganese sulfate, manganese ascorbate, manganese amino acid chelates, manganese aspartate, manganese picolinate, manganese fumarate, manganese malate, manganese succinate, manganese citrate or a mixture thereof.

### Magnesium

In one embodiment, the dosage of magnesium is about 35-350, 200-350 or 300-350 mg/day. A dosage of about 300-350 mg/day may be preferred for breast-feeding women.

In one embodiment, the dosage of magnesium is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

In one embodiment, the dosage of magnesium for a lactating woman is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

In one embodiment, the dosage of magnesium is at least 302 mg/day. In a further embodiment, the dosage of magnesium is at least 305 mg/day. In a still further embodiment, the dosage of magnesium is ranging from 302 to 350 mg/day, for example from 305 to 350 mg/day.

In one embodiment, the dosage of magnesium for a lactating woman is at least 302 mg/day. In a further embodiment, the dosage of magnesium is at least 305 mg/day. In a still further embodiment, the dosage of magnesium is ranging from 302 to 350 mg/day, for example from 305 to 350 mg/day.

The magnesium may be comprised in any form suitable for ingestion by a woman such as a pregnant woman, a woman trying to conceive or a lactating woman. For example, magnesium may be comprised in the form of magnesium chloride, magnesium citrate, magnesium sulfate, magnesium oxide, magnesium hydroxide, magnesium amino acid chelates (e.g. chelates of glycinate, lysinate, orotate, taurate, aspartate, threonate and/or malate) or a mixture thereof.

### Copper

In one embodiment, the dosage of copper is about 0.1-10, 0.1-2 or 0.5-1.5 mg/day.

In one embodiment, the dosage of copper is at least 1.250 mg/day/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

In one embodiment, the dosage of copper for a lactating woman is at least 1.250 mg/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

In one embodiment, the dosage of copper is at least 1.46 mg/day. In a further embodiment, the dosage of copper is at least 1.48 mg/day. In a still further embodiment, the dosage of copper is ranging from 1.46 to 10 mg/day, for example from 1.46 to 2 mg/day, for example from 1.48 to 1.50 mg/day.

In one embodiment, the dosage of copper for a lactating woman is at least 1.46 mg/day. In a further embodiment, the dosage of copper is at least 1.48 mg/day. In a still further embodiment, the dosage of copper is ranging from 1.46 to 10 mg/day, for example from 1.46 to 2 mg/day, for example from 1.48 to 1.50 mg/day.

The copper may be comprised in any form suitable for ingestion by a woman such as a pregnant woman, a woman trying to conceive or a lactating woman. For example, copper may be comprised in the form of copper oxide, copper chloride, copper gluconate, copper sulfate, copper amino acid chelates or a mixture thereof.

### Zinc

In one embodiment, the dosage of zinc may be about 5-40, 7-13 or 9.5-12 mg/day.

In one embodiment, the dosage of zinc is at least 9.5 mg/day. In a further embodiment, the dosage of zinc is at least 10 mg/day. In a still further embodiment, the dosage of zinc is ranging from 9.5 to 12 mg/day, for example from 9.5 to 11.5 mg/day, for example from 10 to 11 mg/day.

In one embodiment, the dosage of zinc for a lactating woman is at least 9.5 mg/day. In a further embodiment, the dosage of zinc is at least 10 mg/day. In a still further embodiment, the dosage of zinc is ranging from 9.5 to 12 mg/day, for example from 9.5 to 11.5 mg/day, for example from 10 to 11 mg/day.

The zinc may be comprised in any form suitable for ingestion by a woman such as a pregnant woman, a woman trying to conceive or a lactating woman. For example, zinc may be comprised in the form of zinc acetate, zinc chloride, zinc citrate, zinc gluconate, zinc lactate, zinc oxide, zinc sulfate, zinc carbonate or a mixture thereof.

### Selenium

In one embodiment, the dosage of selenium may be about 20-400, 25-250, 26-85 or 60-70 µg/day.

In one embodiment, the dosage of selenium is at least 131 µg/day. In a further embodiment, the dosage of selenium is at least 135 µg/day. In a still further embodiment, the dosage of selenium is ranging from 131 to 400 µg/day, for example from 140 to 250 µg/day, for example from 150 to 200 µg/day/day.

In one embodiment, the dosage of selenium for a lactating woman is at least 131 µg/day. In a further embodiment, the dosage of selenium is at least 135 µg/day. In a still further embodiment, the dosage of selenium is ranging from 131 to 400 µg/day, for example from 140 to 250 µg/day, for example from 150 to 200 µg/day.

The selenium may be comprised in any form suitable for ingestion by a woman such as a pregnant woman, a woman trying to conceive or a lactating woman. For example, selenium may be comprised in the form of sodium selenite, sodium hydrogen selenite or a mixture thereof.

### Calcium

In one embodiment, the dosage of calcium is about 100-2500, 500-2000 or 1000-1500 mg/day.

In one embodiment, the dosage of calcium is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day.

In one embodiment, the dosage of calcium for a lactating woman is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day

In one embodiment, the dosage of calcium is at least 860 mg/day. In a further embodiment, the dosage of calcium is at least 900 mg/day. In a still further embodiment, the dosage of calcium is ranging from 860 to 2500 mg/day, for example from 900 to 2000 mg/day, for example from 900 to 1500 mg/day.

In one embodiment, the dosage of calcium for a lactating woman is at least 860 mg/day. In a further embodiment, the dosage of calcium is at least 900 mg/day. In a still further embodiment, the dosage of calcium is ranging from 860 to 2500 mg/day, for example from 900 to 2000 mg/day for example from 900 to 1500 mg/day.

The calcium may be comprised in any form suitable for ingestion by a woman such as a pregnant woman, a woman trying to conceive or a lactating woman. For example, calcium may be comprised in the form of calcium citrate, calcium carbonate or a mixture thereof.

### Phosphorus

In one embodiment, the dosage of phosphorous is about 70-4000, 100-1500 or 250-1250 mg/day.

In one embodiment, the dosage of phosphorus is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

In one embodiment, the dosage of phosphorus for a lactating woman is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

In one embodiment, the dosage of phosphorus is at least 1250 mg/day. In a further embodiment, the dosage of phosphorus is at least 1275 mg/day. In a still further embodiment, the dosage of phosphorus is ranging from 1250 to 4000 mg/day, for example from 1275 to 2000 mg/day, for example from 1300 to 1500 mg/day.

In one embodiment, the dosage of phosphorus for a lactating woman is at least 1250 mg/day. In a further embodiment, the dosage of phosphorus is at least 1275 mg/day. In a still further embodiment, the dosage of phosphorus is ranging from 1250 to 4000 mg/day, for example from 1275 to 2000 mg/day, for example from 1300 to 1500 mg/day.

The phosphorous may be comprised in any form suitable for ingestion by a woman such as a pregnant woman, a woman trying to conceive or a lactating woman. For example, phosphorous may be comprised in the form of sodium phosphate.

In a further embodiment of the present invention, the dosage of iron is ranging from 9.5 to 60 mg/day, for example from 9.5 to 30 mg/day, for example 9.5 to 20 mg/day; the dosage of managese is ranging from 2.1 to 4 mg/day, for example from 2.3 to 3.5 mg/day; the dosage of magnesium is ranging from 270 to 350 mg/day, for example from 300 to 350 mg/day; the dosage of copper is ranging from 1.250 to 10 mg/day, for example from 1.30 to 2 mg/day for example from 1.30 to 1.50 mg/day; the dosage of calcium is ranging from 750 to 2500 mg/day, for example from 850 to 2000 mg/day, for example from 900 to 1500 mg/day; and the dosage of phosphorus is ranging from 1300 to 4000 mg/day, for example from 1300 to 2000 mg/day, for example from 1300 to 1500 mg/day. In such embodiment, the subject receiving the mineral combination or composition comprising it is for example a lactating woman.

### n-3 Fatty Acids

In another aspect, the invention provides a food parameter which is an n-3 fatty acid for use in treating or preventing mastitis in a subject, preferably wherein the fatty acid is selected from the group consisting of docosahexaenoic acid (DHA), 18:3 n-3 octadecatrienoic acid (alpha-linolenic acid), Eicosapentaenoic acid (EPA) and mixtures thereof.

### Protein

In another aspect, the invention provides a food parameter which is protein selected from the group consisting of alphalactalbumin, lactoferrin and albumin for use in treating or preventing mastitis, for example subclinical mastitis, in a subject.

Such agents (vitamin E, n-3 fatty acids, alphalactalbumin, lactoferrin and albumin) may be used in any form suitable for ingestion by animals, preferably humans (e.g. are non-toxic). The agents may be used, for example in compositions such as nutritional compositions, in any appropriate amount. The skilled person will be able to determine appropriate amounts depending on the desired dosage of the agent. Dosages may depend on factors such as the age, size and health status of the woman to whom they are administered, on her lifestyle, as well as on her genetic heritage. Dosages may be in line with the recommended daily allowance (RDA) developed by organisations such as the Food and Nutrition Board of the National Academy of Sciences.

### Vitamin E

In one embodiment, the dosage of vitamin E is about 11-1000, 7.5-300 or 11-19 mg/day.

In one embodiment, the dosage of vitamin E is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day for example from 9.5 to 19 mg/day.

In one embodiment, the dosage of vitamin E for a lactating woman is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day, for example from 9.5 to 19 mg/day.

The vitamin E may be, for example, in the form of a tocopherol or a mixture of different tocopherols. For example, the vitamin E may be alpha-tocopherol, gamma-tocopherol or a mixture of alpha-tocopherol and gamma-tocopherol.

The vitamin E may be comprised in any form suitable for ingestion by a woman such as a pregnant woman, a woman trying to conceive or a lactating woman, for example, alpha-tocopherol and/or gamma-tocopherol, and/or may be comprised in the form of tocopherol concentrate mix, _{L}-vitamin E, _{D},_{L}-vitamin E, tocopherols mixed pure, _{D},_{L}-alpha-tocopherol, _{D},_{L}-alpha tocopheryl acetate, tocopherol rich extract or a mixture thereof.

In one embodiment, the vitamin E is alpha-tocopherol.

In one embodiment, the dosage of docosahexaenoic acid (DHA) is less than or equal to 1000 mg/day, preferably about 500-1000 mg/day.

In one embodiment, the dosage of alpha-linolenic acid is less than or equal to 2000 mg/day, preferably about 500-1000 mg/day.

In one embodiment, the dosage of phosphatidylcholine is about 1500-1750 mg/day.

In one embodiment, the dosage of lecithin is about 1500-1750 mg/day.

In one embodiment, the dosage of lactoferrin is about 5-500 mg/day, preferably about 100-500 mg/day.

With respect to dosages defined herein as amounts per daily dose the amount of nutrient in a composition administered to the subject may vary depending upon whether it is intended to be consumed once a day, or more or less frequently.

### EXAMPLES

### Example 1

### Methods

### Study Population

This study used data from ‘ATLAS’, a longitudinal, observational study across seven European countries between December 2012 and January 2016 ¹. The study was approved by the institutional and local ethical boards for each center and was registered at ClincalTrials.gov with identifier NCT01894893. Maternal and infant demographics, anthropometry, and medical history were collected by trained and certified research nurses and assistants. Written informed consent was obtained from all women in their respective local languages.

Human breastmilk was collected from 305 women in 7 European countries. Of those, 185 provided information on dietary intake. 8 women were further excluded, resulting in a final sample size of 177 women.

### SCM Analysis

Milk samples were obtained using an electric breast pump (Medela Symphony, Switzerland) from the same breast throughout the study period, at 11:00 h ± 2:00 h to avoid circadian influence. Samples were first frozen at -18° C. until delivery to the Nestlé Research Centre (Lausanne, Switzerland) and then at -80° C. for further analysis.

SCM was assessed in early lactation, at visits 1 (V1) between 0-3 days postpartum, visit 2 (V2) around 17 days postpartum ± 3 days, and at visit 3 (V3) around 30 days postpartum ± 3 days. SCM was defined as having a sodium potassium ratio (Na/K) in breastmilk higher than 0.6 at any of the three visits. Moderate SCM was defined as Na/K ratio between >0.6 and <_ 1 while severe SCM as Na/K ratio > 1.

### Dietary Intake Data

Dietary intake was assessed with 3-day food diaries at V2 and V3. The dietary information was then translated to nutrient and food group intakes by Nutrilog using the French food group classification and nutrient composition database (CIQUAL).

Diets which contained less than 1074.8 kcal or more than 4776.9 kcal of energy were considered outliers. After removing the outlier diets from the dataset, we considered each visit in V2-V3 in turn and the subset of subjects who attended that visit. For each visit, we removed a subject and all associated dietary information from a given visit if fewer than two non-outlier diets were reported for that subject across the three-day survey period for that visit. For example, if a subject S1 attended visit V2 and reported one outlier diet and one non-outlier diet for that visit, then we removed S1 and all her reported diets for V2 from the dataset. 177 subjects who attended at least one visit were retained for analysis, which we will refer to as the "filtered dataset.

Dietary reference values for nutrients intake for lactating women were extracted from the European Food Safety Authority (EFSA)’s summary report on Dietary Reference Values for nutrients ².

### Dietary Inflammatory Index method

'The procedure used to calculate the DIETARY INFLAMMATORY INDEX scores has been previously described. Briefly, the DIETARY INFLAMMATORY INDEX was developed after a comprehensive review of peer-reviewed literature published from 1950 to 2010. A total of 1943 qualifying articles that assessed the role of 45 food macronutrients, micronutrients, flavonoids and individual food items on interleukins (IL-1B, IL-4, IL-6, IL-10), Tumor Necrosis Factor-alpha (TNF-a) and high-sensitivity C-Reactive Protein (CRP). The inflammatory potential for each food parameter was scored according to whether it increased (+1), had no effect (0) or decreased (-1) the above-mentioned inflammatory biomarkers.^{’4}

Of the 45 food parameters to create the DIETARY INFLAMMATORY INDEX score, the inventors were able to use 27 as the following ones could not be measured with the food diaries used in this study (excluded food parameters were: Caffeine, eugenol, garlic, ginger, onion, saffron, trans fat, turmeric, green/black tea, flavan-3-ol, flavones, flavonols, flavonones, anthocyanidins, isoflavones, pepper, thyme/oregano, and rosemary).

For the analyses, the inventors followed steps 6, 7, and 8 described in FIG. 1 of the Shivappa paper (Reference FIG. 1 in the drawings of the present patent application). First, they calculated the nutrient z-score for each of the 27 nutrients based on the world average and standard deviation shown in Table 2 of the Shivappa paper (which is also here below reported as Reference Table 2). Then, they calculated percentiles for each nutrient. Each percentile score was then doubled and ‘1’ was subtracted in order to achieve a symmetrical distribution with values centred on 0 (null) and bounded between 21 (maximally antiinflammatory) and 11 (maximally pro-inflammatory). For step 7, they multiplied the centered percentile value for each food parameter by its respective ‘overall food parameter-specific inflammatory effect score’ from Table 2 of the Shivappa publication to obtain the ‘food parameter-specific DII score’. Finally, all of the ‘food parameter-specific Dietary Inflammatory Index scores’ were summed to create the ‘overall Dietary Inflammatory Index score’ for each individual in our study (step 8 in FIG. 1 of the paper).

### Statistical Analyses

We used multivariable and logistic regression to examine the association between nutrient intake and Dietary Inflammatory Index in relation to SCM and continuous Na/K ratio. We used Kruskall test for examining the association between Dietary Inflammatory Index and SCM and Mann Whitney U-test for DIETARY INFLAMMATORY INDEX and SCM severity. Reference Table 2

Table 2

Food parameters included in the dietary inflammatory index, inflammatory effect scores, and intake values from the global composite data set; Dietary Inflammatory Index Development Study, Columbia, SC, USA, 2011-2012

| Food parameter | Weighted number of articles | Raw inflammatory effect score* | Overall inflammatory effect score^{†} | Global daily mean intake^{‡} (units/d) | so^{‡} |
|---|---|---|---|---|---|
| Alcohol (g) | 417 | -0.278 | -0.278 | 13.98 | 3.72 |
| Vitamin B₁₂ (µg) | 122 | 0.205 | 0.106 | 5.15 | 2.70 |
| Vitamin B₆ (mg) | 227 | -0.379 | -0.365 | 1.47 | 0.74 |
| β-Carotene (µg) | 401 | -0.584 | -0.584 | 3718 | 1720 |
| Caffeine (g) | 209 | -0.124 | -0.110 | 8.05 | 6.67 |
| Carbohydrate (g) | 211 | 0.109 | 0.097 | 272.2 | 40.0 |
| Cholesterol (mg) | 75 | 0.347 | 0.110 | 279.4 | 51.2 |
| Energy (kcal) | 245 | 0.180 | 0.180 | 2056 | 338 |
| Eugenol (mg) | 38 | -0.868 | -0.140 | 0.01 | 0.08 |
| Total fat (g) | 443 | 0.298 | 0.298 | 71.4 | 19.4 |
| Fibre (g) | 261 | -0.663 | -0.663 | 18.8 | 4.9 |
| Folic acid (µg) | 217 | -0.207 | -0.190 | 273.0 | 70.7 |
| Garlic (g) | 277 | -0.412 | -0.412 | 4.35 | 2.90 |
| Ginger (g) | 182 | -0.588 | -0.453 | 59.0 | 63.2 |
| Fe (mg) | 619 | 0.032 | 0.032 | 13.35 | 3.71 |
| Mg (mg) | 351 | -0.484 | -0.484 | 310.1 | 139.4 |
| MUFA (g) | 106 | 0.019 | -0.009 | 27.0 | 6.1 |
| Niacin (mg) | 58 | 1.000 | -0.246 | 25.90 | 11.77 |
| n-3 Fatty acids (g) | 2588 | 0.436 | -0.436 | 1.06 | 1.06 |
| n-6 Fatty acids (g) | 924 | 0.159 | -0.159 | 10.80 | 7.50 |
| Onion (g) | 145 | -0.490 | -0.301 | 35.9 | 18.4 |
| Protein (g) | 102 | 0.049 | 0.021 | 79.4 | 13.9 |
| PUFA (g) | 4002 | -0.337 | -0.337 | 13.88 | 3.76 |
| Riboflavin (mg) | 22 | -0.727 | -0.068 | 1.70 | 0.79 |
| Saffron (g) | 33 | -1.000 | -0.140 | 0.37 | 1.78 |
| Saturated fat (g) | 205 | 0.429 | 0.373 | 28.6 | 8.0 |
| Se (µg) | 372 | -0.191 | -0.191 | 67.0 | 25.1 |
| Thiamin (mg) | 65 | -0.354 | -0.098 | 1.70 | 0.66 |
| Trans fat (g) | 125 | 0.432 | 0.229 | 3.15 | 3.75 |
| Turmeric (mg) | 814 | 0.785 | -0.785 | 533.6 | 754.3 |
| Vitamin A (RE) | 663 | -0.401 | -0.401 | 983.9 | 518.6 |
| Vitamin C (mg) | 733 | -0.424 | -0.424 | 118.2 | 43.46 |
| Vitamin D (µg) | 996 | -0.446 | -0.446 | 6.26 | 2.21 |
| Vitamin E (mg) | 1495 | 0.419 | -0.419 | 8.73 | 1.49 |
| Zn (mg) | 1036 | -0.313 | 0.313 | 9.84 | 2.19 |
| Green/black tea (g) | 735 | -0.536 | 0.536 | 1.69 | 1.53 |
| Flavan-3-ol (mg) | 521 | -0.415 | -0.415 | 95.8 | 85.9 |
| Flavones (mg) | 318 | -0.616 | -0.616 | 1.55 | 0.07 |
| Flavonols (mg) | 887 | -0.467 | -0.467 | 17.70 | 6.79 |
| Flavonones (mg) | 65 | -0.908 | -0.250 | 11.70 | 3.82 |
| Anthocyanidins (mg) | 69 | -0.449 | -0.131 | 18.05 | 21.14 |
| Isoflavones (mg) | 484 | -0.593 | -0.593 | 1.20 | 0.20 |
| Pepper(g) | 78 | - 0.397 | -0.131 | 10.00 | 7.07 |
| Thyme/oregano (mg) | 24 | -1.000 | 0.102 | 0.33 | 0.99 |
| Rosemary (mg) | 9 | -0.333 | 0.013 | 1.00 | 15.00 |

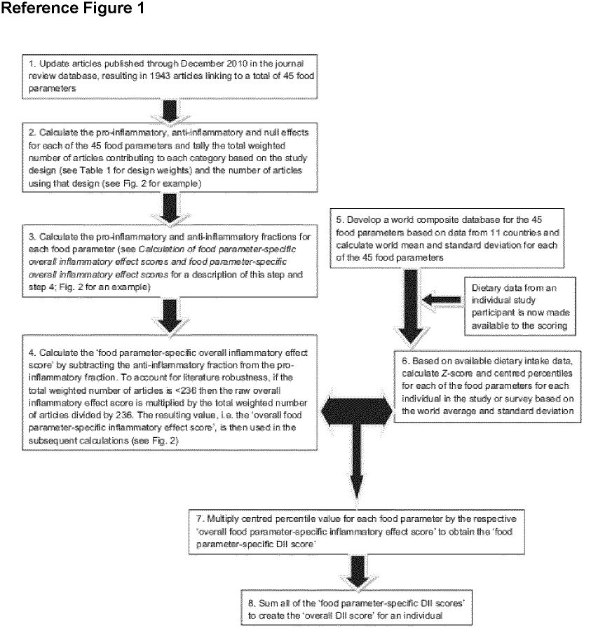

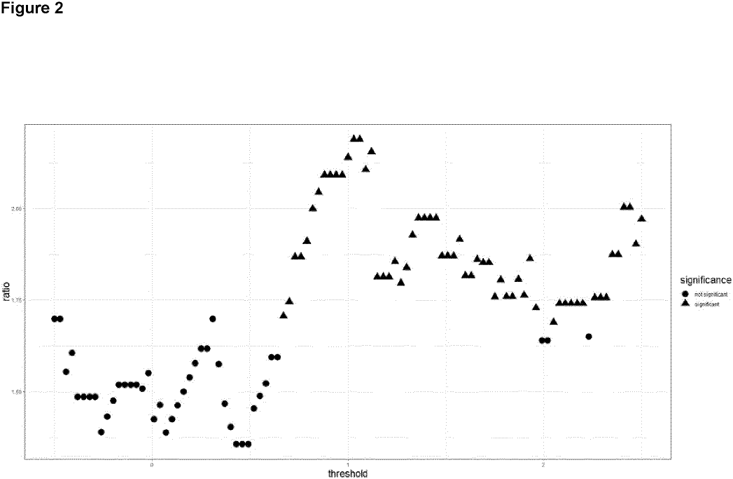

RE, retinol equivalents.
*This is referred to as the ‘food parameter-specific raw inflammatory effect score’ in the text and is abbreviated here for ease of presentation. Note that the effect is per unit amount noted for each food parameter.
†This refers to the ‘food parameter- specific overall inflammatory effect score’ accounting for the robustness of the literature, which is considered optimal at the median of 236 articles, and is computed as described in the text and illustrated for saturated fat in FIG. 2.
‡From the world composite database, as described in the text.

### Results

Table 1 reports the median Dietary Inflammatory Index value for the diet of women with SCM (i.e. those with human milk sodium potassium (Na/K) ratio >0.6 during any of the following visits: days 2, 17, and 30), and for women with no SCM (i.e. those with no SCM are defined as having a Na/K ratio <_0.6 during any of the following visits: days 2, 17, and 30).

Table 3 reports the median Dietary Inflammatory Index value for the diet of women with severe SCM (i.e. those with human milk sodium potassium (Na/K) ratio >1 during any of the following visits: days 2, 17, and 30), of women with moderate SCM (i.e. those with human milk sodium potassium (Na/K) ratio comprised between 0.6 and 1 during any of the following visits: days 2, 17, and 30), and of women with no SCM (i.e. those with no SCM are defined as having a Na/K ratio <_0.6 during any of the following visits: days 2, 17, and 30).

Table 1

| | Median Dietary Inflammatory Index | Mean Dietary Inflammatory Index | Count | Percentage |
|---|---|---|---|---|
| No SCM | 0,61 | 0,55 | 125 | 71 |
| Any SCM*: Na/K ratio > 0.6 | 1,67 | 1,28 | 52 | 29 |

Table 3

Dietary Inflammatory Index in relation to occurrence and severity of Sub-Clinical Mastitis (SCM)*

| | Median Dietary Inflammatory Index | Mean Dietary Inflammatory Index | Count | Percentage |
|---|---|---|---|---|
| No SCM | 0,61 | 0,55 | 125 | 71 |
| Moderate SCM | 1,24 | 1,04 | 31 | 18 |
| Severe SCM | 1,96 | 1,64 | 21 | 12 |

* SCM diagnosis at any visit (days 2, 17 and 30). taking maximum Na/K ratio at all visits to classify patient.

These data show that the diet of women with subclinical mastitis (irrespective of whether severe or moderate) is characterized by a higher Dietary Inflammatory Indexas compared to the diet of women not having subclinical mastitis.

These findings thus provide evidence that consuming foods and/or following a diet characterized by a low Dietary Inflammatory Index may prevent and/or treat subclinical mastitis in lactating women as well as prevent its evolution into mastitis. These finding also indicate that inflammatory potential in the diet of lactating women (thus ultimately the Dietary Inflammatory Index of their diet) is a risk indicator for the occurrence of SCM and that accordingly Dietary Inflammatory Index of the diet may be used as a risk prediction tool for SCM in this population.

Table 4 here below reported further details the incidence of SCM in tertiles of the study population based on Dietary Inflammatory Index scores. The cutoff point for the tertiles are the following: 0.15 (cutoff between T1 and T2) and 1.68 (cutoff between T2 and T3).

Table 4

Dietary Inflammatory Index score tertiles

| | T1 | T2 | T3 |
|---|---|---|---|
| Sample size | 59 | 59 | 59 |
| Median | -1.09 | 0.78 | 2.62 |
| Min | -3.59 | 0.17 | 1.73 |
| Max | 0.15 | 1.68 | 3.97 |
| SCM* (Na/K ratio > 0.6) | 13 | 14 | 25 |
| Proportion of SCM* by tertile | 0.22 | 0.24 | 0.42 |
| No SCM | 46 (37%) | 45 (36%) | 34 (27%) |
| Moderate | 10 (32%) | 8 (26%) | 13 (42%) |
| Severe SCM | 3 (14%) | 6 (29%) | 12 (57%) |

FIG. 2 graphically maps the Dietary Inflammatory Index cut off values at the X axis and the relative risk of SCM incidence based on this cutoff point at the Y axis. For example, at a cutoff point of 1, 41% of women above this cutoff have SCM, while 19% of women below this cutoff have SCM. The relative risk is then 2.14 meaning there is 2.14 more risk of getting SCM if a woman is above this cutoff point.

The inventors observed that beyond a Dietary inflammatory index of 0.8 the relative risk doubles in a statistically significant manner.

The inventors also observed that for Dietary Inflammatory Index values beyond 1.00, the relative risk of occurrence for subclinical mastitis is 2.14.

The inventors further observed that for Dietary Inflammatory Index values beyond 1.68, the relative risk of occurrence for subclinical mastitis is 1.95 statistically significant via chi-square test (p=0.006)]

### Example 2

### Impact of Intake of Specific Individual Nutrients within a Category with Food Parameter-Specific Overall Inflammatory Effect Score Lower than 0 (n-3 fatty fatty acids) on Dietary Inflammatory Index Score

In order to investigate the impact on the Dietary inflammatory Index score (and consequently on the risk of developing mastitis and /or subclinical mastitis) of specific nutrients within a category associated with a food parameter-specific overall inflammatory effect score lower than 0, the Mean/median daily intake of some exemplary nutrients was calculated within tertiles of the study population based on Dietary Inflammatory Index scores (same as reported in Table 4 of Example 1).

Results are reported in Table 5 here below:

Table 5

| | Tertiles of DIETARY INFLAMMATORY INDEX | | | |
|---|---|---|---|---|
| | T1 | T2 | T3 | p values |
| Fatty acid 20:5 omega-3 (EPA) | 0.08 | 0.04 | 0.03 | 0.00 |
| Fatty acid 22:6 omega-3 (DHA) | 0.13 | 0.07 | 0.06 | 0.01 |

T1 happens to be the tertile associated with the highest intakes of EPA and DHA in the study population. As T1 is also the tertile associated with lower incidence of subclinical mastitis and a lower Dietary Inflammation Index Score, it results that increasing the intake of nutrients having a food parameter-specific overall inflammatory effect score lower than 0 (such as DHA, ALA and EPA, falling under the category of n-3 fatty acids) is beneficial for prevention and/or treatment of subclinical mastitis.

### Example 3

### Provision of Level of Risk of Developing a Subclinical Mastitis Based on Daily Dietary Intake

An individual’s dietary intake is captured daily by getting all food items’ intake with quantities. This food intake is broken down into nutrients via a standard FCT (CIQAL in this example). For each of the 45 food parameters, the individual’s nutrient intake is normalized via the global mean and Standard deviation as declared in Shivappa et al, Public Health Nutrition, 17 (8), 1689-1696. For example, if the individual’s Energy intake is 1517.6 kcal and the global mean and SD are respectively 2056 kcal and 338 kcal, the normalized score (z-score) would be -1.5929 ((1517.6-2056)/338). This z-score is converted as a percentile to the normal distribution (centred at 0 and with SD 1) and is centred to zero by multiplying by 2 and subtracting 1. In this example this gives:

Percentile(-1.5929) = 0.0556 and then the centered value would be -0.8888 ( = (2 x 0.0556) -1). This centered value is then multiplied to the OIES (overall food parameter specific inflammatory effect score) from Table 2 of Shivappa et al, Public Health Nutrition, 17 (8), 1689-1696 and would give -0.15999 (-0.8888 x 0.18). This will be the food parameter Dietary Inflammatory Index score. Summing up all the available food parameter Dietary Inflammatory Index score would give the overall Dietary Inflammatory Index score which is then associated to a level of risk of developing subclinical mastitis as follows:Overall Dietary inflammatory Index score < 0.68 is associated to a low risk level;

Overall Dietary inflammatory Index score > 0.68 but <1.68 is associated to a medium risk level;

Overall Dietary inflammatory Index score > 1.68 is associated to a high risk level.

All publications mentioned in the above specification are herein incorporated by reference. Various modifications and variations of the disclosed agents, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for the prevention and/or treatment of mastitis comprising administering a diet with an overall Dietary Inflammatory index score lower than 0.8 to a subject in need of same.

2. Method for prevention and/or treatment of mastitis in a lactating woman, comprising administering to such subject a diet which has an overall Dietary Inflammatory index score lower than 0.8.

3-4. (canceled)

5. Method to assess the risk of developing mastitis in a subject, the method comprising:
a) recording dietary intake data of the subject, ;
b) calculate the overall Dietary Inflammatory Index score using the dietary intake data recorded in step a);
c) associating to such overall Dietary Inflammatory Index score a level of risk of developing mastitis, for the subject; and
d) recommending to the subject to follow a diet with an overall Dietary inflammatory index score lower than 0.8 and/or to consume food parameters, or compositions comprising them, having a food parameter-specific overall inflammatory effect score lower than 0; and/or
e) recommending to the subject to limit the consumption of food parameters, or compositions comprising them, having a food parameter-specific overall inflammatory effect score higher than 0.

6. Method according to claim 5, which is implemented on a computer.

7. Method according to claim 5 wherein step b) is performed by: calculating Z-scores and centred percentiles for each of the food parameters for each subject, for example a lactating woman, based on the world average and standard deviation, then multiplying centred percentile value for each food parameter by the respective overall food parameter specific inflammatory effect score to obtains the food parameter specific Dietary Inflammatory Index score, then sum all of the food parameter-specific Dietary Inflammatory Index scores to create the overall Dietary inflammatory Index score for the subject, .

8. Method according to claim 5, wherein in step c) the level of risk for the subject of developing mastitis, is:
low risk if the subject has an overall Dietary Inflammatory Index score lower than 0.8;
medium risk if the subject has an overall Dietary Inflammatory Index score comprised between 0.8 and 1.68; and
high risk if the subject has an overall Dietary Inflammatory Index score higher than 1.68.

9. Method according to claim 5, wherein if in step c) the risk for the subject is medium or high, in steps d) and e) it is recommended to the subject:
to follow a diet with an overall Dietary inflammatory index score lower than 0.8; and/or
to consume food parameters, or compositions comprising them, having a food parameter-specific overall inflammatory effect score lower than 0, for example selected in the group consisting of: alcohol, Vitamin B6, beta-carotene, caffeine, carbohydrates, fibers, folic acid, garlic, ginger, magnesium, n-3 fatty acids, n-6 fatty acids, onion, Polyunsaturated fatty acids, Vitamin B2, saffron, Selenium, Vitamin B 1, turmeric, Vitamin A, Vitamin C, Vitamin D, Vitamin E, Zinc, green and/or black tea, flavan-3-ol, flavones, flavonols, flavonones, antocyanidins, isoflavones, pepper, thyme and/or oregano, rosemary and combinations thereof; and/or
to consume food parameters, or composition comprising them, which are selected in the list consisting of: potassium, Vitamin B5, Vitamin B12, Manganese, copper, calcium, phosphorus, iron, phosphatidyl choline, alpha-lactalbumin, lactoferrin, albumin,n-3 fatty acids, especially DHA and/or EPA, and combinations thereof; and
to limit the consumption of food parameters, or compositions comprising them, having a food parameter-specific overall inflammatory effect score higher than 0.

10-12. (canceled)
